(19) United States
(12) Patent Application Publication
(10) Pub. No.: US 2023/0405322 A1
(43) Pub. Date: Dec. 21, 2023

(54) ELECTRICAL STIMULATION HAIR GROWING DEVICE

(71) Applicant: Nazhiyuan Technology (Tangshan) Co., Ltd., Tangshan (CN)

(72) Inventor: Charles HSU, Tangshan (CN)

(73) Assignee: Nazhiyuan Technology (Tangshan) Co., Ltd., Tangshan (CN)

(21) Appl. No.: 18/460,856

(22) Filed: Sep. 5, 2023

Related U.S. Application Data
(63) Continuation-in-part of application No. PCT/CN2022/076834, filed on Feb. 18, 2022.
Application No. PCT/CN2022/076834 is a continuation-in-part of application No. PCT/CN2022/076936, filed on Feb. 18, 2022.

(30) Foreign Application Priority Data
Mar. 4, 2021 (CN) 202110238030.6
Mar. 4, 2021 (CN) 202120464858.9
Sep. 27, 2021 (CN) 202111133404.4
Sep. 27, 2021 (CN) 202122343493.7

(51) Int. Cl. A61N 1/36; A61N 1/04; A61H 23/02; H02N 1/04; H02N 2/00

(52) U.S. Cl. CPC A61N 1/36034; A61N 1/0464; A61N 1/0484; A61H 23/02; H02N 1/04; H02N 2/00; A61H 2201/10; A61H 2201/0107; A61H 2205/021; A61H 2201/0165; A61H 2201/123; A61H 2201/165

(57) ABSTRACT

An electrical stimulation hair growing device includes: an energy providing module, an electrical pulse generating module and an electrical stimulation module. The energy providing module provides electric or mechanical energy to the electrical pulse generating module respectively through electrical or mechanical connection, the electrical pulse generating module being in electrical connection to the electrical stimulation module, and the electrical stimulation module being in contact with a skin surface of an area in need of hair growing; and the electrical pulse generating module generates regular electrical pulse signals by means of the electric or mechanical energy and transmits the regular electrical pulse signals to the electrical stimulation module, so that the electrical stimulation module applies the electrical pulse signals to the area in need of hair growing for electrical stimulation.

### CROSS-REFERENCE TO RELATED APPLICATIONS

This application is a continuation-in-part of International Application No. PCT/CN2022/076834, filed on Feb. 18, 2022, which claims priority to Chinese Patent Application No. 202110238030.6, filed on Mar. 4, 2021, and Chinese Patent Application No. 202120464858.9, filed on Mar. 4, 2021; and also a continuation-in-part of International Application No. PCT/CN2022/076936, filed on Feb. 18, 2022, which claims priority to Chinese Patent Application No. 202111133404.4, filed on Sep. 27, 2021, and Chinese Patent Application No. 202122343493.7, filed on Sep. 27, 2021. All of the aforementioned applications are incorporated herein by reference in their entireties.

### TECHNICAL FIELD

The present disclosure relates to the technical field of biomedicine, in particular to an electrical stimulation hair growing device.

### BACKGROUND

Currently, hair loss has gradually turned into a topic of concern. Hair loss happens for various reasons, such as long-term extreme fatigue and anxiety under excessive mental stress, or congenital genetic factors. Hair loss will not cause serious damages to human body, but it will seriously impact all aspects of people's daily life. Therefore, it is urgent in modern society to treat hair loss.

As for the problem of hair loss, although the electrical stimulation hair growing devices based on triboelectricity generating technology in the prior art can be used without external power supply, they cannot accurately apply different electrical pulses for electrically stimulating hair growing specific to depilation or hair loss conditions of different objects in need of hair growing, nor can take into account energy storage, charging and other functions.

Therefore, in the prior art, an electrical stimulation hair growing device capable of accurately applying electrical pulses for electrically stimulating hair growing is lacking.

### SUMMARY

As for the afore-mentioned problem, the present disclosure discloses an electrical stimulation hair growing device, so as to solve or at least partly solve the afore-mentioned problem.

In order to realize the afore-mentioned object, the technical solution below is adopted in the present disclosure.

The present disclosure discloses an electrical stimulation hair growing device, comprising: an energy providing module, an electrical pulse generating module and an electrical stimulation module, where:
the energy providing module provides electric or mechanical energy to the electrical pulse generating module respectively through electrical or mechanical connection, the electrical pulse generating module being in electrical connection to the electrical stimulation module, and the electrical stimulation module being in contact with a skin surface of an area in need of hair growing; and
the electrical pulse generating module generates regular electrical pulse signals by means of the electric or mechanical energy and transmits the regular electrical pulse signals to the electrical stimulation module, so that the electrical stimulation module applies the electrical pulse signals to the area in need of hair growing for electrical stimulation.

Further, the energy providing module comprises a vibration module, where:
the vibration module is used for generating vibration with a predetermined frequency and a predetermined amplitude; and
the electrical pulse module is provided in contact with the vibration module, and is used for converting vibration acted thereon by the vibration module into electrical pulses and outputting the electrical pulses, and/or for converting mechanical energy acted thereon by external environment into electrical pulses and outputting the electrical pulses.

Further, the vibration module further comprises: a power module, a driver module, and at least one vibration part; where:
the power module is connected to the driver module, and is used for providing electric energy to the driver module;
the driver module is connected to the at least one vibration part, and is used for controlling the at least one vibration part to vibrate at the predetermined frequency and the predetermined amplitude; and
the at least one vibration part is provided in contact with the electrical pulse module, and is used for generating vibration having the predetermined frequency and the predetermined amplitude so as to act the vibration on the electrical pulse module.

Further, the power module further comprises a battery, a vibration switch module and a power management module; where:
the battery is connected to the vibration switch module, and is used for providing electric energy to the power management module through the vibration switch module;
the vibration switch module is connected to the power management module, and is used for controlling whether the battery provides electric energy to the power management module; and
the power management module is connected to the driver module, and is used for converting the electric energy output by the battery through the vibration switch module, so as to provide electric energy to the driver module.

Further, the electrical stimulation hair growing device further comprises an electrical stimulation switch module; where: the electrical stimulation switch module is respectively connected to the electrical pulse module and the electrical stimulation module, and is used for enabling the electrical pulse module to output electrical pulses to the electrical stimulation module when the electrical pulse module and the electrical stimulation module are conducted by the electrical stimulation switch module.

Further, the electrical stimulation hair growing device further comprises an electrical stimulation switch module and a charging module; where: the electrical stimulation switch module is respectively connected to the electrical pulse module and the electrical stimulation module, and is used for enabling the electrical pulse module to output electrical pulses to the electrical stimulation module when the electrical pulse module and the electrical stimulation module are conducted by the electrical stimulation switch module; the charging module is respectively connected to the electrical stimulation switch module and the power module, and is used for preprocessing the electrical pulses output by the electrical pulse module when the electrical pulse module and the charging module are conducted by the electrical stimulation switch module, and for outputting the preprocessed electrical pulses to the power module for charging.

Further, a transmission insulating part for transmitting vibration and for avoiding conduction of the at least one vibration part and the electrical pulse module is further provided between the at least one vibration part and the electrical pulse module.

Further, the vibration part is a rotor motor, a longitudinal linear motor or a transverse linear motor.

Further, the number of vibration parts is multiple, where: the multiple vibration parts comprise the same number of the longitudinal linear motors and the transverse linear motors, and the longitudinal linear motors and the transverse linear motors are uniformly distributed in arrays on the electrical pulse module.

Further, the energy providing module comprises a power supply module and a central control module, where:
the power supply module provides power to the central control module and the electrical pulse generating module respectively by means of electrical connection; and
the electrical pulse generating module generates regular electrical pulse signals by means of control of the central control module and transmits the regular electrical pulse signals to the electrical stimulation module.

Further, the central control module comprises a parameter adjustment module; where:
the parameter adjustment module is in electrical connection to the electrical pulse generating module; and
the parameter adjustment module is used for adjusting at least one of voltage, current, waveform, amplitude, width and frequency of the electrical pulse signal generated by the electrical pulse generating module.

Further, the device comprises a remote control module, and the central control module comprises a wireless transceiver module; where:
the wireless transceiver module is in wireless connection to the remote control module, and the wireless transceiver module is in electrical connection to the parameter adjustment module; and
the wireless transceiver module enables the parameter adjustment module to make adjustment according to an instruction from the remote control module, and controls the electrical pulse generating module to generate corresponding regular electrical pulse signals which are transmitted to the electrical stimulation module.

Further, the power supply module comprises a charging module and an energy storage module; where:
the energy storage module is in electrical connection respectively to the charging module, the central control module and the electrical pulse generating module; and
the energy storage module receives electric energy through the charging module, and provides power to the central control module and the electrical pulse generating module.

Further, the device comprises a wearable object capable of electricity self-generating, and the power supply module comprises a preprocessing module; where:
the preprocessing module is in electrical connection respectively to the wearable object capable of electricity self-generating and the energy storage module; and
the wearable object capable of electricity self-generating is used for converting force acted thereon into electrical signals and transmitting the electrical signals to the preprocessing module, and the preprocessing module preprocesses received electrical signals and transmits preprocessed electrical signals to the energy storage module.

Further, the electrical stimulation module further comprises a plurality of electrodes; and at least one electrical stimulation bulge is provided on a side surface where the plurality of electrodes is in contact with the area in need of hair growing.

Further, the electrical pulse module comprises: a triboelectric generator, and/or a piezoelectric generator, and/or a pressure inductive cable based on a triboelectric generator and/or a piezoelectric generator, and/or a piezoelectric and triboelectric hybrid generator.

Further, the triboelectric generator is a triboelectricity generating film; and/or the piezoelectric generator is a piezoelectricity generating film.

Further, the triboelectric generator comprises a friction part-based knitted triboelectric generator; where:
the friction part-based knitted triboelectric generator comprises: at least one first friction part and at least one second friction part; where:
the at least one first friction part and the at least one second friction part are knitted with each other to form the friction part-based knitted triboelectric generator, and at least one of the at least one first friction part and the at least one second friction part comprises a friction interface capable of contacting friction; and
the at least one first friction part and/or the at least one second friction part have an electrical signal output end of the friction part-based knitted triboelectric generator.

Further, the pressure inductive cable based on a triboelectric generator and/or a piezoelectric generator is knitted to form the electrical pulse module having a predetermined shape.

Further, the at least one first friction part and the at least one second friction part are knitted to form the electrical pulse module having a predetermined shape.

Further, the predetermined shape is a hat or cap shape, a headgear shape, a turban shape, a collar shape or a sheet shape.

Further, the electrical stimulation hair growing device further comprises a wearable body; the wearable body is worn on an object in needed of hair growing, and is provided with the at least one vibration module, the electrical pulse module and/or the electrical stimulation module.

Further, the wearable body is a hat or cap, a headgear, a turban, a collar or a sheet.

Further, the electrical stimulation hair growing device is a detachable and/or portable electrical stimulation hair growing device.

Following are advantages and beneficial effects of the present disclosure.

The electrical stimulation hair growing device of the present disclosure is capable of providing energy to the electrical stimulation module through mechanical or electric energy, so that the electrical pulse module generates electrical pulses and applies the electrical pulses to the electrical stimulation module for electrical stimulation.

The electrical stimulation hair growing device, which is realized by means of providing mechanical energy to the electrical pulse module, can produce the following effects.

Firstly, the electrical stimulation hair growing device provided in the present disclosure generates vibration with a predetermined frequency and a predetermined amplitude through the vibration module, and accurately controls the electrical pulses output by the electrical pulse module to the electrical stimulation module, so as to accurately apply different electrical pulses to different objects in need of hair growing, thereby activating epidermal hair follicles and promoting hair growth.

Secondly, since a triboelectric generator, and/or a piezoelectric generator, and/or a pressure inductive cable based on a triboelectric generator and/or a piezoelectric generator, and/or a piezoelectric and triboelectric hybrid generator are adopted in the electrical pulse module, the electrical stimulation hair growing device provided in the present disclosure can directly convert the mechanical energy in external environment into electrical pulses when there is no or insufficient external power supply, so that electrical stimulation can be performed by the electrical stimulation module, which is energy-efficient and environmentally friendly, and can also avoid the failure to use the electrical stimulation hair growing device caused by the absence of external power supply.

Thirdly, the electrical stimulation hair growing device provided in the present disclosure can convert the mechanical energy in external environment into electric energy by means of the electrical pulse module, so as to charge the power module, which can prolong the power supply time of the power module, as well as is energy-efficient and environmentally friendly.

Fourthly, since a triboelectric generator, and/or a piezoelectric generator, and/or a pressure inductive cable based on a triboelectric generator and/or a piezoelectric generator, and/or a piezoelectric and triboelectric hybrid generator are adopted in the electrical pulse module, the electrical stimulation hair growing device provided in the present disclosure is light and small, and is easy for users to carry and/or use.

Lastly, the electrical stimulation hair growing device provided in the present disclosure is simple in structure and manufacturing process, and has low cost, so it is suitable for large-scale industrial production.

The electrical stimulation hair growing device, which is realized by means of providing electric energy to the electrical pulse module, can produce the following effects.

Firstly, in the electrical stimulation hair growing device provided in the present disclosure, the electrical pulse generating module is accurately controlled by the central control module to generate regular electrical pulse signals and transmit regular electrical pulse signals to the electrical stimulation module, so that the electrical stimulation module applies the electrical pulse signals to the area in need of hair growing for electrical stimulation, thereby activating epidermal hair follicles of a human head and promoting hair growth.

Secondly, the remote control module is used in the present disclosure for remote intelligent control, which enables the electrical stimulation hair growing device to be used more flexibly.

Lastly, the wearable object capable of electricity self-generating is used in the present disclosure for energy storage and charging of the power supply module, so that electrical stimulation can still be realized by the electrical stimulation hair growing device even in case of insufficient external power supply, which enables the electrical stimulation hair growing device to be used in many different scenes; and the electrical stimulation hair growing device provided in the present disclosure is simple in structure and manufacturing process, and has low cost, so it is suitable for large-scale industrial production.

### BRIEF DESCRIPTION OF THE DRAWINGS

All other advantages and beneficial effects will be clear and apparent for ordinary technicians in this field after reading detailed description of the preferable embodiments below. The drawings are included only to show the preferable embodiments, and should not be regarded as limits of the present disclosure. Besides, a single reference symbol represents the same part in all drawings. In the drawings:

FIG. 1 is a structure diagram showing the electrical stimulation hair growing device in one example of the present disclosure;

FIG. 2 is a diagram showing a circuit module of the vibration module in one example of the present disclosure;

FIG. 3 is a diagram showing another circuit module of the vibration module in one example of the present disclosure;

FIG. 4 is a structure diagram showing at least one first friction part of the friction part-based knitted triboelectric generator in one example of the present disclosure;

FIG. 5 is another structure diagram showing at least one first friction part of the friction part-based knitted triboelectric generator in one example of the present disclosure;

FIG. 6 is a structure diagram showing an electrode of the electrical stimulation module in an example of the present disclosure;

FIG. 7 is another structure diagram showing the electrical stimulation hair growing device in one example of the present disclosure;

FIG. 8 is a diagram showing a circuit module of the electrical stimulation hair growing device in another example of the present disclosure;

FIG. 9 is a structure diagram showing the electrical stimulation hair growing device in one example of the present disclosure;

FIG. 10 is a structure diagram showing the electrical stimulation hair growing device in one example of the present disclosure;

FIG. 11 is a structure diagram showing the electrical stimulation hair growing device in one example of the present disclosure;

FIG. 12 is a diagram showing a wearable object capable of electricity self-generating in one example of the present disclosure;

FIG. 13 is a diagram showing another wearable object capable of electricity self-generating in one example of the present disclosure; and

FIG. 14 is a structure diagram showing the electrical stimulation hair growing device in one example of the present disclosure.

### DETAILED DESCRIPTION OF THE EMBODIMENTS

In order to make the object, the technical solution and the advantages of the present disclosure clearer, the technical solution of the present disclosure will be clearly and completely illustrated with reference to examples and corresponding drawings of the present disclosure. Obviously, the examples illustrated are merely part of the examples of the present disclosure, rather than all. Based on the examples of the present disclosure, all other examples obtained by one skilled in the art without paying creative efforts belong to the scope of protection of the present disclosure.

With reference to the drawings, technical solutions in all examples of the present disclosure will be illustrated in detail hereinafter.

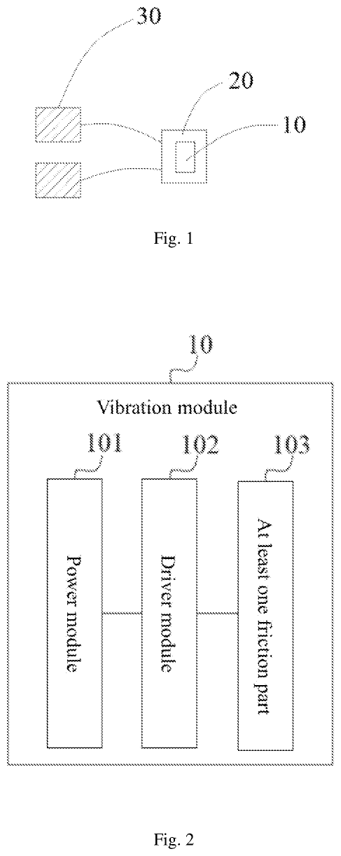

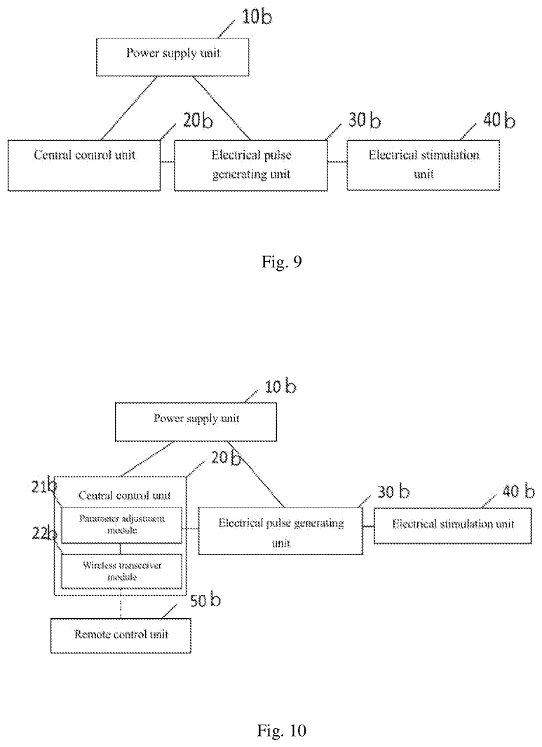

On the one hand, the present disclosure provides an electrical stimulation hair growing device. The electrical stimulation hair growing device comprises: an energy providing module, an electrical pulse generating module and an electrical stimulation module. The energy providing module provides electric or mechanical energy to the electrical pulse generating module respectively through electrical or mechanical connection, the electrical pulse generating module being in electrical connection to the electrical stimulation module, and the electrical stimulation module being in contact with a skin surface of an area in need of hair growing; and the electrical pulse generating module generates regular electrical pulse signals by means of the electric or mechanical energy and transmits the regular electrical pulse signals to the electrical stimulation module, so that the electrical stimulation module applies the electrical pulse signals to the area in need of hair growing for electrical stimulation. In the afore-mentioned electrical stimulation hair growing device, there are many ways for the energy providing module to provide energy to the electrical pulse generating module, for example, by providing mechanical energy (FIGS. 1 to 8), and, for another example, by providing electric energy (FIGS. 9 to 14). Following are respective detailed introductions.

FIG. 1 is a structure diagram showing Example 1 of the electrical stimulation hair growing device provided in the present disclosure. As shown in FIG. 1, the electrical stimulation hair growing device of Example 1 comprises: a vibration module 10, an electrical pulse module 20 and an electrical stimulation module 30. The vibration module 10 is used for generating vibration with a predetermined frequency and a predetermined amplitude; the electrical pulse module 20 is provided in contact with the vibration module 10, and is used for converting vibration acted thereon by the vibration module 10 into electrical pulses and outputting the electrical pulses, and/or for converting mechanical energy acted thereon by external environment into electrical pulses and outputting the electrical pulses; and the electrical stimulation module 30 is provided on an area in need of hair growing (not shown), is connected to the electrical pulse module 20 and is used for acting the electrical pulses output by the electrical pulse module 20 on the area in need of hair growing for electrical stimulation.

Further, as shown in FIGS. 1 and 2, the vibration module 10 may comprise: a power module 101, a driver module 102, and at least one vibration part 103. The power module 101 is connected to the driver module 102, and is used for providing electric energy to the driver module 102; the driver module 102 is connected to the at least one vibration part 103, and is used for controlling the at least one vibration part 103 to vibrate at the predetermined frequency and the predetermined amplitude; and the at least one vibration part 103 is provided in contact with the electrical pulse module 20, and is used for generating vibration having the predetermined frequency and the predetermined amplitude so as to act the vibration on the electrical pulse module 20.

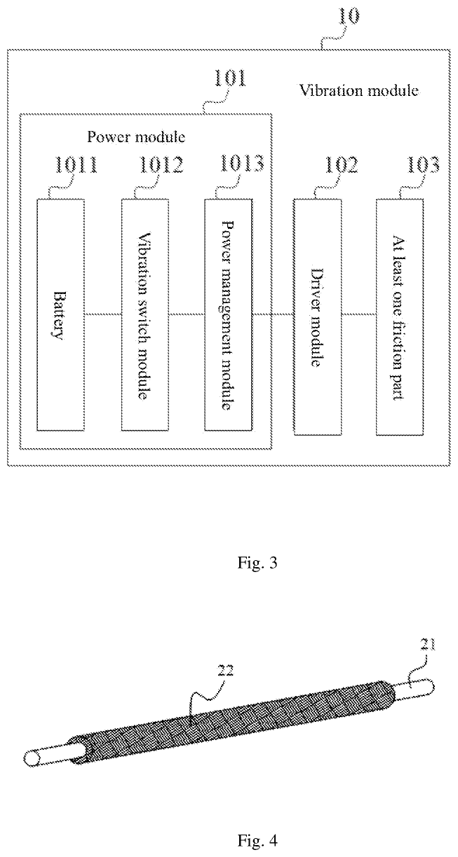

Optionally, as shown in FIGS. 1 and 3, the power module 101 further comprises a battery 1011, a vibration switch module 1012 and a power management module 1013. The battery 1011 is connected to the vibration switch module 1012, and is used for providing electric energy to the power management module 1013 through the vibration switch module 1012; the vibration switch module 1012 is connected to the power management module 1013, and is used for controlling whether the battery 1011 provides electric energy to the power management module 1013; and the power management module 1013 is connected to the driver module 102, and is used for converting the electric energy output by the battery 1011 through the vibration switch module 1012, so as to provide electric energy to the driver module 20.

In Example 1, the battery 1011 can be a battery in the prior art, and the type thereof can be selected by one skilled in the art according to actual necessities and will not be limited here. For example, the battery 1011 can be a rechargeable or non-rechargeable nickel-cadmium battery, nickel-metal hydride battery, lithium-ion battery, or lead-acid battery.

The vibration switch module 1012 can be a switch in the prior art. For example, the vibration switch module 1012 can be a key switch, a touch switch or a film switch, and the type thereof can be selected by one skilled in the art according to actual necessities and will not be limited here. Since film switches are light, thin and flexible, which can increase the comfort of use, the vibration switch module 1012 is preferably a film switch.

Specifically, when the vibration switch module 1012 is turned on, the vibration switch module 1012 conducts the battery 1011 and the power management module 1013, and the battery 1011 provides electric energy to the power management module 1013 through the vibration switch module 1012; when the vibration switch module 1012 is turned off, the vibration switch module 1012 disconnects the battery 1011 from the power management module 1013, and the battery 1011 stops providing electric energy to the power management module 1013 through the vibration switch module 1012.

The function of the power management module 1013 is to convert the electric energy output by the battery 1011 through the vibration switch module 1012 into a voltage suitable for the driver module 102. Therefore, the power management module 1013 can be a power management chip in the prior art capable of converting the electric energy output by the battery 1011 through the vibration switch module 1012 into a voltage suitable for the driver module 102, which will not be limited here.

The driver module 102 can be a driver chip in the prior art, and the type thereof can be selected by one skilled in the art according to actual necessities and will not be limited here.

The at least one vibration part 103 is a vibration part in the prior art, and the type thereof can be selected by one skilled in the art according to actual necessities and will not be limited here. For example, the at least one vibration part 103 is preferably a rotor motor, a longitudinal linear motor or a transverse linear motor. In addition, the number of the at least one vibration part 103 may be one or more, which can be selected by one skilled in the art according to actual necessities and will not be limited here.

In a preferable embodiment, the number of vibration parts 103 is multiple, and the multiple vibration parts 103 comprise the same number of longitudinal linear motors and transverse linear motors, which are uniformly distributed in arrays on the electrical pulse module 20. This way of arrangement is to enable the multiple vibration parts 103 to apply longitudinal and transverse vibration to the electrical pulse module 20, so as to accurately control the predetermined frequency and predetermined amplitude of the electrical pulses output by the electrical pulse module 20.

It should be understood that one or more longitudinal linear motors can be provided in contact with the electrical pulse module 20 if it is required to apply only longitudinal vibration to the electrical pulse module 20 through the at least one vibration part 103, and one or more lateral linear motors can be provided in contact with the electrical pulse module 20 if it is required to apply only transverse vibration to the electrical pulse module 20 through the at least one vibration part 103.

In the present disclosure, as the number of the vibration parts 103 increases, the intensity (i.e., the amplitude) of the vibration applied to the electrical pulse module 20 also increases, namely, there are two ways of increasing intensity of the vibration applied to the electrical pulse module 20: one is to increase the vibration intensity applied to the electrical pulse module 20 through the driver module 102, and the other one is to increase the vibration intensity applied to the electrical pulse module 20 by providing a plurality of vibration parts 103. These two ways can be selected by one skilled in the art according to actual necessities, and will not be limited here.

The providing position of the vibration module 10 can be selected by one skilled in the art according to actual necessities, and will not be limited here. However, it must be sure that the vibration generated by the vibration module 10 can be applied to the electrical pulse module 20.

In an optional embodiment, as shown in FIG. 1, the vibration module 10 is provided in contact with the electrical pulse module 20, so that the vibration generated by the vibration module can be better applied to the electrical pulse module 20. The vibration module 10 can be provided in direct or indirect contact with the electrical pulse module 20. In addition, the vibration module is provided in indirect contact with the electrical pulse module 20 to avoid conduction of the vibration module 10 and the electrical pulse module 20. Therefore, a transmission insulating part (not shown), which is used for transmitting vibration and avoiding conduction of the vibration module 10 and the electrical pulse module 20, is further provided between the vibration module and the electrical pulse module 20. The transmission insulating part can be selected from insulating films in the prior art, such as polyvinyl chloride film, polyethylene film, polypropylene film, polystyrene film, etc.

Specifically, if the vibration module 10 comprises a power module 101, a driver module 102 and at least one vibration part 103, any or more of the power module 101, the driver module 102 and the at least one vibration part 103 can be provided in direct or indirect contact with the electrical pulse module 20; however, it must be sure that the vibration generated by the at least one vibration part 103 can be applied to the electrical pulse module 20. Preferably, the at least one vibration part 103 is provided in direct or indirect contact with the electrical pulse module 20; moreover, in order to avoid conduction of the at least one vibration part 103 and the electrical pulse module 20, a transmission insulating part (not shown), which is used for transmitting vibration and avoiding conduction of the at least one vibration part 103 and the electrical pulse module 20, is further provided between the at least one vibration part 103 and the electrical pulse module 20. The transmission insulating part can be selected from insulating films in the prior art, such as polyvinyl chloride film, polyethylene film, polypropylene film, polystyrene film, etc.

In this example, as for the types of the power management module 1013, the driver module 102 and the at least one vibration part 103, one skilled in the art can select the power management chip, the driver chip and the vibration part in the prior art according to actual necessities, which will not be limited here. However, it should be noted that, when selecting types of the power management module 1013, the driver module 102 and the at least one vibration part 103, one skilled in the art should ensure that parameters of the three match with each other. For example, when the driving voltage of the driver module 102 is a DC voltage of 3.3 V, it is necessary that the power management module 1013 can output a DC voltage of 3.3 V. Similarly, when the starting voltage of the at least one vibration part 103 is a DC voltage of 2.3 V, it is necessary that the driver module 102 can output a DC voltage of 2.3 V.

The electrical pulse module 20 in Example 1 can be provided on an object in need of hair growing, such as a human body, an animal body and other organisms that need hair growing. When vibration is generated by the vibration module 10 or the mechanical energy from external environment is acted on the object in need of hair growing, reciprocating extrusion or loosening occurs between the electrical pulse module 20 and the object in need of hair growing, and relative motion is generated due to relative friction, and then is converted into electrical pulses and is output. However, it should be understood that the electrical pulse module 20 can also be provided on other objects than the object in need of hair growing. For example, the electrical pulse module 20 can be provided on a table, and vibration can be applied by the vibration module 10 or by hand tapping or the like, so that the electrical pulse module 20 generates electrical pulses.

Further, the electrical pulse module 20 may comprise a triboelectric generator, and/or a piezoelectric generator, and/or a pressure inductive cable based on a triboelectric generator and/or a piezoelectric generator, and/or a piezoelectric and triboelectric hybrid generator in the prior art. The type of the electrical pulse module 20 can be selected by one skilled in the art according to actual necessities, and will not be limited here.

The triboelectric generator can be one in the prior art, which has a three-layer structure, a four-layer structure, a five-layer intermediate film structure or a five-layer intermediate electrode structure; the above-mentioned triboelectric generator comprises at least two surfaces constituting friction interfaces, and has an output end; the piezoelectric generator can be one in the prior art, which is made of zinc oxide, PZT, PVDF or other piezoelectric materials.

In a preferable embodiment, the triboelectric generator is a triboelectricity generating film; and/or the piezoelectric generator is a piezoelectricity generating film. The triboelectricity generating film and/or the piezoelectricity generating film therein can be a triboelectricity generating film and/or a piezoelectricity generating film in the prior art, which can be selected by one skilled in the art according to actual necessities and will not be limited here.

In another preferable embodiment, the triboelectric generator comprises a friction part-based knitted triboelectric generator in the prior art. For better understanding by one skilled in the art of the friction part-based knitted triboelectric generator comprised in the triboelectric generator, specific structure of the friction part-based knitted triboelectric generator comprised in the triboelectric generator in the prior art will be introduced in detail.

The friction part-based knitted triboelectric generator comprised in the triboelectric generator comprises: at least one first friction part and at least one second friction part. The at least one first friction part and the at least one second friction part are knitted with each other so as to form the friction part-based knitted triboelectric generator, and the at least one first friction part and/or the at least one second friction part comprise a friction interface capable of contacting friction; and the at least one first friction part and/or the at least one second friction part have an electrical signal output end of the friction part-based knitted triboelectric generator.

In a first embodiment, the friction part-based knitted triboelectric generator comprised in the triboelectric generator comprises: at least one first friction part and at least one second friction part. The at least one first friction part comprises a first polymer insulator; the at least one second friction part comprises a second electric conductor; the first polymer insulator and the second electric conductor are knitted with each other so as to form the friction part-based knitted triboelectric generator; a friction interface is formed or friction interfaces are formed through contacting friction between the first polymer insulator and the second electric conductor, and/or between the first polymer insulator and the object in need of hair growing, and/or between the second electric conductor and the object in need of hair growing; and the second electric conductor serves as an electrical signal output end of the friction part-based knitted triboelectric generator.

In a second embodiment, the friction part-based knitted triboelectric generator comprised in the triboelectric generator comprises: at least one first friction part and at least one second friction part. The at least one first friction part comprises a first electric conductor and a first polymer insulator; the at least one second friction part comprises a second electric conductor; the first polymer insulator is provided on an outside surface of the first electric conductor; the first electric conductor and the first polymer insulator are knitted with the second electric conductor so as to form the friction part-based knitted triboelectric generator; a friction interface is formed or friction interfaces are formed through contacting friction between the first electric conductor and the first polymer insulator, and/or between the first electric conductor and the second electric conductor, and/or between the first electric conductor and the object in need of hair growing, and/or between the first polymer insulator and the second electric conductor, and/or between the first polymer insulator and the object in need of hair growing, and/or between the second electric conductor and the object in need of hair growing; and the first electric conductor and/or the second electric conductor serve as an electrical signal output end of the friction part-based knitted triboelectric generator.

In a third embodiment, the friction part-based knitted triboelectric generator comprised in the triboelectric generator comprises: at least one first friction part and at least one second friction part. The at least one first friction part comprises a first electric conductor and a first polymer insulator; the at least one second friction part comprises a second electric conductor and a second polymer insulator; the first polymer insulator is provided on an outside surface of the first electric conductor, and the second polymer insulator is provided on an outside surface of the second electric conductor; the first electric conductor and the first polymer insulator are knitted with the second electric conductor and the second polymer insulator so as to form the friction part-based knitted triboelectric generator; a friction interface is formed or friction interfaces are formed through contacting friction between the first electric conductor and the first polymer insulator, and/or between the first electric conductor and the second polymer insulator, and/or between the first electric conductor and the object in need of hair growing, and/or between the first electric conductor and the second electric conductor, and/or between the first polymer insulator and the second polymer insulator, and/or between the first polymer insulator and the object in need of hair growing, and/or between the first polymer insulator and the second electric conductor, and/or between the second polymer insulator and the object in need of hair growing; and/or between the second electric conductor and the second polymer insulator, and/or between the second electric conductor and the object in need of hair growing; and the first electric conductor and/or the second electric conductor serve as an electrical signal output end of the friction part-based knitted triboelectric generator.

In a fourth embodiment, the friction part-based knitted triboelectric generator comprised in the triboelectric generator comprises: at least one first friction part and at least one second friction part. The at least one first friction part comprises a first electric conductor and a first polymer insulator; the at least one second friction part comprises a second polymer insulator; the first polymer insulator is provided on an outside surface of the first electric conductor; the first electric conductor and the first polymer insulator are knitted with the second polymer insulator so as to form the friction part-based knitted triboelectric generator; a friction interface is formed or friction interfaces are formed through contacting friction between the first electric conductor and the first polymer insulator, and/or between the first electric conductor and the second polymer insulator, and/or between the first electric conductor and the object in need of hair growing, and/or between the first polymer insulator and the second polymer insulator, and/or between the first polymer insulator and the object in need of hair growing, and/or between the second polymer insulator and the object in need of hair growing; and the first electric conductor serves as an electrical signal output end of the friction part-based knitted triboelectric generator.

In a fifth embodiment, the friction part-based knitted triboelectric generator comprised in the triboelectric generator comprises: at least one first friction part and at least one second friction part. The at least one first friction part comprises a first electric conductor; the at least one second friction part comprises a second electric conductor; the first electric conductor and the second electric conductor are knitted with each other so as to form the friction part-based knitted triboelectric generator; a friction interface is formed or friction interfaces are formed through contacting friction between the first electric conductor and the second electric conductor, and/or between the first electric conductor and the object in need of hair growing, and/or between the second electric conductor and the object in need of hair growing; and the first electric conductor and/or the second electric conductor serve as an electrical signal output end of the friction part-based knitted triboelectric generator.

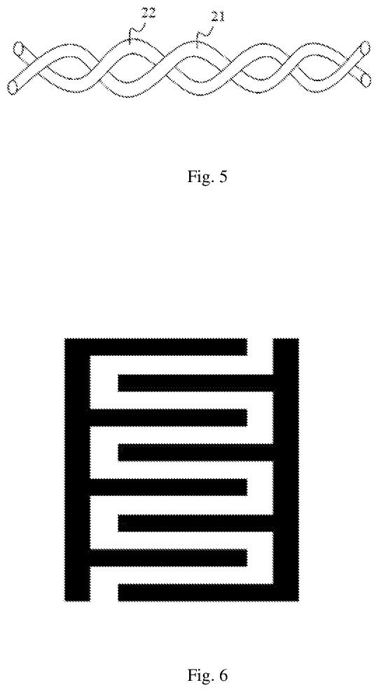

Further, in the afore-mentioned second, third, and fourth embodiments, the first polymer insulator may be provided on the outside surface of the first electric conductor by means of coating or winding and knitting, so as to form the at least one first friction part; specifically, FIG. 4 shows that a first polymer insulating layer 22 is provided on the outside surface of the first electric conductor 21 by means of coating so as to form the at least one first friction part, and FIG. 5 shows that the first polymer insulator 22 is provided on the outside surface of the first electric conductor 21 by means of winding and knitting so as to form the at least one first friction part. In addition, in the afore-mentioned third embodiment, the second polymer insulator may also be provided on the outside surface of the second electric conductor by means of coating or winding and knitting, so as to form the at least one second friction part. In other words, the first polymer insulator is provided on the outside surface of the first electric conductor by means of coating or winding and knitting, so as to form the at least one first friction part; and/or, the second polymer insulator is provided on the outside surface of the second electric conductor by means of coating or winding and knitting, so as to form the at least one second friction part. These can be selected by one skilled in the art according to actual necessities, and will not be limited here.

It should be noted that when the at least one first friction part is formed by means of winding and knitting, the at least one first friction part can be formed by winding and knitting of one first electric conductor 21 with one first polymer insulator 22, as shown in FIG. 5; the at least one first friction part can also be formed by winding and knitting of one first electric conductor with multiple first polymer insulators; the at least one first friction part can also be formed by winding and knitting of multiple first electric conductors with one first polymer insulator; and the at least one first friction part can even be formed by winding and knitting of multiple first electric conductors with multiple first polymer insulators; these can be selected by one skilled in the art according to actual necessities and will not be limited here. For description about forming the at least one second friction part by means of winding and knitting, please refer to the description about forming the at least one first friction part by means of winding and knitting, which will not be repeated here.

Further, in the first embodiment, the first polymer insulator may comprise a plurality of polymer insulated wires forming the first polymer insulator by means of winding and knitting, and/or the second electric conductor may comprise a plurality of conductive wires forming the second electric conductor by means of winding and knitting; in the second embodiment, the first electric conductor may comprise a plurality of conductive wires forming the first electric conductor by means of winding and knitting, and/or the first polymer insulator may comprise a plurality of polymer insulated wires forming the first polymer insulator by means of winding and knitting, and/or the second electric conductor may comprise a plurality of conductive wires forming the second electric conductor by means of winding and knitting; in the third embodiment, the first electric conductor may comprise a plurality of conductive wires forming the first electric conductor by means of winding and knitting, and/or the first polymer insulator may comprise a plurality of polymer insulated wires forming the first polymer insulator by means of winding and knitting, and/or the second electric conductor may comprise a plurality of conductive wires forming the second electric conductor by means of winding and knitting, and/or the second polymer insulator may comprise a plurality of polymer insulated wires forming the second polymer insulator by means of winding and knitting; in the fourth embodiment, the first electric conductor may comprise a plurality of conductive wires forming the first electric conductor by means of winding and knitting, and/or the first polymer insulator may comprise a plurality of polymer insulated wires forming the first polymer insulator by means of winding and knitting, and/or the second polymer insulator may comprise a plurality of polymer insulated wires forming the second polymer insulator by means of winding and knitting; in the fifth embodiment, the first electric conductor may comprise a plurality of conductive wires forming the first electric conductor by means of winding and knitting, and/or the second electric conductor may comprise a plurality of conductive wires forming the second electric conductor by means of winding and knitting; these can be selected by one skilled in the art according to actual necessities, and will not be limited here. Afore-mentioned means of forming the first friction part, the second friction part, the first electric conductor, the first polymer insulator, the second electric conductor and the second polymer insulator can be used in any way of combination, which can be selected by one skilled in the art according to actual necessities and will not be limited here. For example, in the fourth embodiment, the first polymer insulator is formed through winding and knitting of a plurality of polymer insulated wires, and the first polymer insulator coats the outside surface of the first electric conductor, thereby forming the at least one first friction part; the second polymer insulator is formed through winding and knitting of a plurality of polymer insulated wires, thereby forming the at least one second friction part; and the friction part-based knitted triboelectric generator is formed through winding and knitting of the at least one first friction part and the at least one second friction part.

Further, for better contacting friction of the surface constituting the friction interface in the friction part-based knitted triboelectric generator, a bulge structure may be provided on at least one surface constituting the friction interface in the friction part-based knitted triboelectric generator. The types and quantities of the dips and bulges comprised in the bulge structure are not limited in the present application, and can be flexibly provided by one skilled in the art.

In Example 1, materials for the first electric conductor and the second electric conductor are indium tin oxide, graphene, silver nanowire film, metal or alloy. The metal is gold, silver, platinum, palladium, aluminum, nickel, copper, titanium, chromium, selenium, iron, manganese, molybdenum, tungsten or vanadium; and the alloy is aluminum alloy, titanium alloy, magnesium alloy, beryllium alloy, copper alloy, zinc alloy, manganese alloy, nickel alloy, lead alloy, tin alloy, cadmium alloy, bismuth alloy, indium alloy, gallium alloy, tungsten alloy, molybdenum alloy, niobium alloy or tantalum alloy.

In Example 1, the material for the first polymer insulator and the second polymer insulator is any selected from polytetrafluoroethylene, polydimethylsiloxane, polyimide, aniline formaldehyde resin, polyoxymethylene, ethyl cellulose, polyamide, melamine formaldehyde, polyethylene glycol succinate, cellulose, cellulose acetate, polyethylene adipate, poly(diallyl phthalate), fiber (recycled) sponge, polyurethane elastomer, styrene propylene copolymer, styrene-butadiene copolymer, artificial fiber, polymethyl, methacrylate, polyvinyl alcohol, polyester, polyisobutylene, polyurethane flexible sponges, polyethylene terephthalate, polyvinyl butyral, formaldehyde phenol, neoprene, butadiene propylene copolymer, natural rubber, polyacrylonitrile, acrylonitrile vinyl chloride and polyethylene bisphenol carbonates.

In order to improve the effect of triboelectricity generating, it is preferable that different polymer materials are used for the first polymer material surface and the second polymer material surface, which are capable of contacting friction and are comprised in the at least one first friction part and/or the at least one second friction part. For example: in the third and fourth embodiments, since contacting friction may occur between the first polymer insulator and the second polymer insulator, it is preferable that different polymer materials are used for the first polymer insulator and the second polymer insulator, so as to improve the effect of triboelectricity generating.

It should be understood that the friction part-based knitted triboelectric generator is provided on the object in need of hair growing when it is used in the field of electrical stimulation hair growing, and the object in need of hair growing can be replaced with other objects by one skilled in the art when the friction part-based triboelectric generator is used in other fields; this will not be limited here.

To avoid electromagnetic interference from external environment and avoid the influence of external environmental factors (such as temperature, humidity and/or dust, etc.), it is preferable that a triboelectric generator provided with a shielding layer and a protective layer in sequence at outside, and/or a piezoelectric generator, and/or a pressure inductive cable based on a triboelectric generator and/or a piezoelectric generator, and/or a piezoelectric and triboelectric hybrid generator are adopted in the electrical pulse module 20, thereby ensuring normal operation of the electrical pulse module 20.

To prevent the normal operation of the electrical pulse module 20 from being affected by merely the electromagnetic interference of external environment, a triboelectric generator provided with a shielding layer at outside, and/or a piezoelectric generator, and/or a pressure inductive cable based on a triboelectric generator and/or a piezoelectric generator, and/or a piezoelectric and triboelectric hybrid generator may be adopted in the electrical pulse module 20; to prevent the normal operation of the electrical pulse module 20 from being affected by external environmental factors (such as temperature, humidity and/or dust, etc.), a triboelectric generator provided with a protective layer at outside, and/or a piezoelectric generator, and/or a pressure inductive cable based on a triboelectric generator and/or a piezoelectric generator, and/or a piezoelectric and triboelectric hybrid generator may be adopted in the electrical pulse module 20.

To increase the user's comfort level, it is preferable that a triboelectric generator, and/or a piezoelectric generator, and/or a pressure inductive cable based on a triboelectric generator and/or a piezoelectric generator, and/or a piezoelectric and triboelectric hybrid generator, which are flexible, are adopted in the electrical pulse module 20.

Optionally, the number of the triboelectric generator, and/or the piezoelectric generator, and/or the pressure inductive cable based on a triboelectric generator and/or a piezoelectric generator, and/or the piezoelectric and triboelectric hybrid generator comprised in the electrical pulse module 20 may be one or multiple; this can be selected by one skilled in the art according to actual necessities and will not be limited here. When multiple triboelectric generators, and/or piezoelectric generators, and/or pressure inductive cables based on a triboelectric generator and/or a piezoelectric generator, and/or piezoelectric and triboelectric hybrid generators are adopted, the multiple triboelectric generators, and/or piezoelectric generators, and/or pressure inductive cables based on a triboelectric generator and/or a piezoelectric generator, and/or piezoelectric and triboelectric hybrid generators are connected in series or in parallel, and the multiple triboelectric generators, and/or piezoelectric generators, and/or pressure inductive cables based on a triboelectric generator and/or a piezoelectric generator, and/or piezoelectric and triboelectric hybrid generators can be provided by means of tiling and/or stacking; these will not be limited here, and can be selected by one skilled in the art according to actual necessities. Preferably, the multiple triboelectric generators, and/or piezoelectric generators, and/or pressure inductive cables based on a triboelectric generator and/or a piezoelectric generator, and/or piezoelectric and triboelectric hybrid generators are provided in tiled arrays.

The electrical stimulation module 30 comprises a plurality of electrodes. The position and/or size of each electrode therein can be flexibly provided by one skilled in the art according to the position and/or size of the area in need of hair growing, and will not be limited here. However, it should be noted that there must be a potential difference among the electrodes pre-applied with electrical stimulation in the plurality of electrodes comprised in the electrical stimulation module thereby ensuring that electrical pulses can be applied to the area in need of hair growing through the plurality of electrodes for electrical stimulation.

To improve the effect of electrical stimulation, at least one electrical stimulation bulge is provided on the surface at one side of the plurality of electrodes in contact with the area in need of hair growing. The number, size, shape, providing and/or shape formed after providing of the at least one electrical stimulation bulge can be selected by one skilled in the art according to actual necessities, and will not be limited here. Preferably, the plurality of electrical stimulation bulges is provided on the surface at one side of the plurality of electrodes in contact with the area in need of hair growing, and the plurality of electrical stimulation bulges is provided in a circular array. More preferably, a plurality of electrical stimulation bulges matching the size of hair follicles of the object in need of hair growing is provided on the surface at one side of the plurality of electrodes in contact with the area in need of hair growing, and the plurality of electrical stimulation bulges is provided in a circular array. These two preferable ways of providing are more suitable for the distribution and size of hair follicles of the object in need of hair growing, so that the electrical pulses can be better applied to the area in need of hair growing for electrical stimulation.

The plurality of electrodes may be strip-shaped electrodes or interdigitated electrodes as shown in FIG. 6; of course, they may also be in other shapes, which will not be limited here. In addition, the electrical stimulation module 30 may be in direct or indirect contact with the area in need of hair growing, which will not be limited here.

For better understanding of the connection mode of the electrical pulse module 20 and the electrical stimulation module 30 in Example 1 by one skilled in the art, connection modes of the electrical pulse module 20 and the electrical stimulation module 30 will be described in detail below.

When the electrical pulse module 20 comprises two output ends between which there is a potential difference, and the electrical stimulation module 30 comprises two electrodes, the two output ends comprised in the electrical pulse module 20 can be respectively and correspondingly connected to the two electrodes comprised in the electrical stimulation module 30; it is also possible that either of the two output ends comprised in the electrical pulse module 20 is selected to be connected to either of the two electrodes comprised in the electrical stimulation module 30, while the other electrode comprised in the electrical stimulation module 30 is directly connected to the object in need of hair growing or other reference potential points.

When the electrical pulse module 20 comprises two output ends between which there is no potential difference (namely, the two output ends are conducted or the potentials thereof are the same), and the electrical stimulation module 30 comprises two electrodes, either of the two output ends comprised in the electrical pulse module 20 may be selected to be connected to either of the two electrodes comprised in the electrical stimulation module 30, while the other electrode comprised in the electrical stimulation module 30 is directly connected to the object in need of hair growing or other reference potential points.

When the electrical pulse module 20 comprises one output end and the electrical stimulation module 30 comprises two electrodes, the one output end comprised in the electrical pulse module 20 may be connected to either of the two electrodes comprised in the electrical stimulation module 30, while the other electrode comprised in the electrical stimulation module 30 is directly connected to the object in need of hair growing or other reference potential points. That is to say, regardless of the connection mode, it must make sure that a potential difference can be generated between the electrodes comprised in the electrical stimulation module 30 for pre-applied electrical stimulation. Cases are similar for other situations and will not be repeated here.

In Example 1, when the electrical pulse module 20 comprises a friction part-based knitted triboelectric generator or a pressure inductive cable based on a triboelectric generator and/or a piezoelectric generator, the at least one first friction part and the at least one second friction part can be used after being knitted by using an existing knitting method into a friction part-based knitted triboelectric generator having a predetermined shape, or the pressure inductive cable based on a triboelectric generator and/or a piezoelectric generator can be used after being knitted into a predetermined shape. For example, one first friction part and one second friction part are knitted by plain knitting, or two pressure inductive cables based on a triboelectric generator and/or a piezoelectric generator are knitted by plain knitting, so as to form an electrical pulse module 20 having a hat or cap shape, which can be used after being provided at an area in need of hair growing on the head of an object in need of hair growing; and the electrical stimulation module 30 is provided on an inside surface of the electrical pulse module 20 close to the object in need of hair growing.

The predetermined shape referred to in Example 1 can be any shape, for example, it can be a hat or cap shape, and can be a headgear shape, a turban shape or a collar shape, and can also be a sheet shape, etc.; this can be selected by one skilled in the art according to actual necessities, and will not be limited here.

Of course, after the at least one first friction part and the at least one second friction part are knitted into a predetermined shape and/or after the pressure inductive cable based on a triboelectric generator and/or a piezoelectric generator is knitted into a predetermined shape, they can also be used in combination with any one or more of other generators referred to in Example 1; this will not be limited here, and can be selected by one skilled in the art according to actual necessities.

Optionally, a protective layer (not shown) of the hair growing device is further provided outside the vibration module 10, the electrical pulse module 20 and/or the electrical stimulation module 30, and is used for protecting the electrical pulse module 20 and/or the electrical stimulation module 30.

The material used for the protective layer of the hair growing device is selected from the materials for polymer insulators.

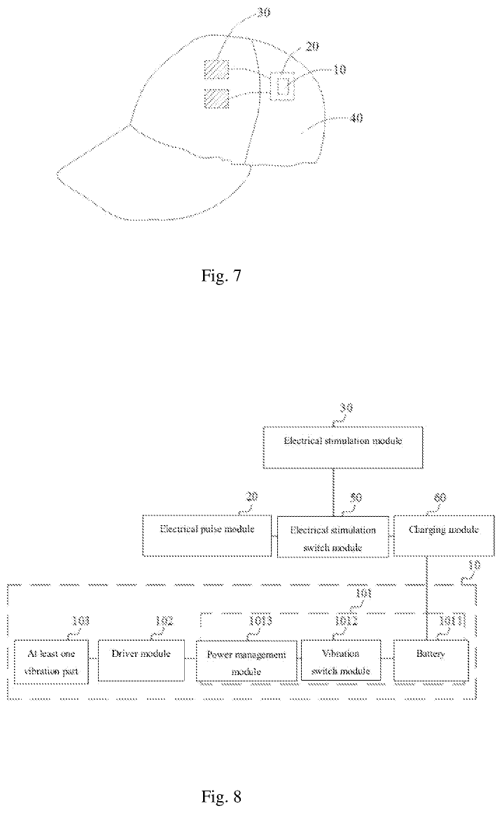

In addition, as shown in FIG. 7, the electrical stimulation hair growing device in Example 1 further comprises: a wearable body 40. The wearable body 40 is worn on the object in need of hair growing (not shown), and is provided with the vibration module 10, the electrical pulse module 20 and/or the electrical stimulation module 30. When the wearable body 40 is worn on the object in need of hair growing, the vibration generated by the vibration module 10 and/or the mechanical energy from external environment are acted on the object in need of hair growing, reciprocating extrusion or loosening will occur between the electrical pulse module 20 and the object in need of hair growing, and relative motion will be generated due to relative friction. The relative motion is converted into electrical pulses before it is output.

When the electrical stimulation hair growing device in Example 1 comprises the wearable body 40, the vibration module 10, the electrical pulse module 20 and/or the electrical stimulation module 30 can be provided on the inside surface, the outside surface and/or the interlayer of the wearable body, and this can be selected by one skilled in the art according to actual necessities and will not be limited here; besides, the wearable body 40 is a wearable object in the prior art, such as: a hat or cap, a headgear, a turban, a collar, or a sheet, etc., which can be selected by one skilled in the art according to actual necessities and will not be limited here. Specifically, the wearable body 40 in FIG. 7 is a cap, and the vibration module 10, the electrical pulse module 20 and the electrical stimulation module 30 are respectively provided on the inside surface of the cap top.

In Example 1, an electrical stimulation switch module (not shown) may be further provided between the electrical pulse module 20 and the electrical stimulation module 30, and is used for controlling whether the electrical pulse module 20 outputs electrical pulses to the electrical stimulation module 30. In other words, the electrical stimulation switch module is respectively connected to the electrical pulse module 20 and the electrical stimulation module 30. When the electrical stimulation switch module is turned on, the electrical pulse module 20 and the electrical stimulation module 30 are conducted through the electrical stimulation switch module, and the electrical pulse module 20 outputs electrical pulses to the electrical stimulation module 30 for electrical stimulation; when the electrical stimulation switch module is turned off, the electrical stimulation switch module disconnects the electrical pulse module 20 from the electrical stimulation module 30, and the electrical pulse module 20 stops outputting electrical pulses to the electrical stimulation module 30 for electrical stimulation.

The electrical stimulation switch module can be a switch in the prior art. For example, the electrical stimulation switch module can be a key switch, a touch switch or a film switch, and the type thereof can be selected by one skilled in the art according to actual necessities and will not be limited here. Since film switches are light, thin and flexible, which can increase the comfort level of use, the electrical stimulation switch module is preferably a film switch.

The electrical stimulation hair growing device provided in Example 1 generates vibration with a predetermined frequency and a predetermined amplitude through the vibration module, and accurately controls the electrical pulses output by the electrical pulse module to the electrical stimulation module, so as to accurately apply different electrical pulses to different objects in need of hair growing, thereby activating epidermal hair follicles and promoting hair growth; besides, since a triboelectric generator, and/or a piezoelectric generator, and/or a pressure inductive cable based on a triboelectric generator and/or a piezoelectric generator, and/or a piezoelectric and triboelectric hybrid generator are adopted in the electrical pulse module, it is possible to directly convert the mechanical energy in external environment into electrical pulses when there is no or insufficient external power supply, so that electrical stimulation can be performed by the electrical stimulation module, which is energy-efficient and environmentally friendly, and can also avoid the failure to use the electrical stimulation hair growing device caused by the absence of external power supply; moreover, the electrical stimulation hair growing device provided in Example 1 is simple in structure and manufacturing process, and has low cost, so it is suitable for large-scale industrial production.

FIG. 8 is a diagram showing a circuit module in Example 2 for the electrical stimulation hair growing device provided in the present disclosure. As shown in FIG. 8, the difference between the electrical stimulation hair growing device in Example 2 and that in Example 1 is that the electrical stimulation hair growing device in Example 2 further comprises: an electrical stimulation switch module 50 and a charging module 60. The electrical stimulation switch module 50 is respectively connected to the electrical pulse module 20 and the electrical stimulation module 30, and is used for enabling the electrical pulse module 20 to output electrical pulses to the electrical stimulation module 30 when the electrical pulse module 20 and the electrical stimulation module 30 are conducted by the electrical stimulation switch module 50; the charging module 60 is respectively connected to the electrical stimulation switch module 50 and the power module 101, and is used for preprocessing the electrical pulses output by the electrical pulse module 20 when the electrical pulse module 20 and the charging module 60 are conducted by the electrical stimulation switch module 50, and for outputting the preprocessed electrical pulses to the power module 101 for charging.

When the power module 101 comprises the battery 1011, the vibration switch module 1012 and the power management module 1013, the charging module 60 is respectively connected to the electrical stimulation switch module 50 and the battery 1011, and is used for preprocessing the electrical pulses output by the electrical pulse module 20 when the electrical pulse module 20 and the charging module 60 are conducted by the electrical stimulation switch module 50, and for outputting the preprocessed electrical pulses to the battery 1011 for charging.

Optionally, the charging module 60 comprises a rectification module (not shown), an amplification module (not shown) and a filtration module (not shown); the rectification module is connected to the electrical stimulation switch module 50, and is used for rectifying the electrical pulses output by the electrical pulse module 20 through the electrical stimulation switch module the amplification module is connected to the rectification module, and is used for amplifying the electrical signals output by the rectification module; and the filtration module is respectively connected to the amplification module and the power module 101, and is used for filtering out interference noise in the electrical signal output by the amplification module, so that the power module 101 is charged.

Specifically, when the power module 101 comprises the battery 1011, the vibration switch module 1012 and the power management module 1013, the charging module 60 comprises a rectification module, an amplification module and a filtration module. The rectification module is connected to the electrical stimulation switch module 50, and is used for rectifying the electrical pulses output by the electrical pulse module 20 through the electrical stimulation switch module the amplification module is connected to the rectification module, and is used for amplifying the electrical signals output by the rectification module; and the filtration module is respectively connected to the amplification module and the battery 1011 in the power module 101, and is used for filtering out interference noise in the electrical signal output by the amplification module, so that the battery 1011 in the power module 101 is charged.

It should be noted that the rectification module, the amplification module and the filtration module comprised in the charging module 60 are all optional modules, which can be selected by one skilled in the art according to actual necessities and will not be limited here. For example, the amplification module can be omitted if amplification is not required. In addition, the rectification module, the amplification module and the filtration module comprised in the charging module 60 all adopt rectification modules, amplification modules and filtration modules in the prior art, which will not be limited here.

In the second embodiment, the electrical stimulation switch module 50 is respectively connected to the electrical pulse module 20, the electrical stimulation module 30 and the charging module 60. When the electrical stimulation switch module 50 is turned on, the electrical pulse module 20 and the stimulation module 30 are conducted through the electrical stimulation switch module 50, and the electrical pulse module 20 outputs electrical pulses to the electrical stimulation module 30 for electrical stimulation; and, when the electrical stimulation switch module 50 is turned off, the electrical stimulation switch module 50 conducts the electrical pulse module 20 and the charging module 60, and the electrical pulses output by the electrical pulse module 20 are output to the power module 101 after being preprocessed by the charging module 60, so that the power module 101 is charged.

More specifically, when the power module 101 comprises the battery 1011, the vibration switch module 1012 and the power management module 1013, the electrical stimulation switch module 50 is respectively connected to the electrical pulse module 20, the electrical stimulation module 30 and the charging module 60. When the electrical stimulation switch module 50 is turned on, the electrical pulse module 20 and the stimulation module 30 are conducted through the electrical stimulation switch module 50, and the electrical pulse module 20 outputs electrical pulses to the electrical stimulation module 30 for electrical stimulation; and, when the electrical stimulation switch module 50 is turned off, the electrical stimulation switch module 50 conducts the electrical pulse module 20 and the charging module 60, and the electrical pulses output by the electrical pulse module 20 are output to the battery 1011 after being preprocessed by the charging module 60, so that the battery 1011 is charged.

The electrical stimulation switch module 50 can be a switch in the prior art, for example, the electrical stimulation switch module 50 can be a single-pole double-throw key switch or the like; the type thereof can be selected by one skilled in the art according to actual necessities and will not be limited here.

In Example 2, the electrical pulse module 20 charges the battery 1011 in the power module 101 through the charging module 60, so the battery 1011 adopts a rechargeable nickel-cadmium battery, nickel-metal hydride battery, lithium-ion battery, or lead-acid battery in the prior art, and will not be limited here.

Except for the afore-mentioned differences, please refer to the description of the electrical stimulation hair growing device in Example 1 for other description of the electrical stimulation hair growing device in Example 2, which will not be repeated here.

The electrical stimulation hair growing device provided in Example 2 generates vibration with a predetermined frequency and a predetermined amplitude through the vibration module, and accurately controls the electrical pulses output by the electrical pulse module to the electrical stimulation module, so as to accurately apply different electrical pulses to different objects in need of hair growing, thereby activating epidermal hair follicles and promoting hair growth; besides, since a triboelectric generator, and/or a piezoelectric generator, and/or a pressure inductive cable based on a triboelectric generator and/or a piezoelectric generator, and/or a piezoelectric and triboelectric hybrid generator are adopted in the electrical pulse module, it is possible to directly convert the mechanical energy in external environment into electrical pulses when there is no or insufficient external power supply, so that electrical stimulation can be performed by the electrical stimulation module, which is energy-efficient and environmentally friendly, and can also avoid the failure to use the electrical stimulation hair growing device caused by the absence of external power supply; meanwhile, the electrical stimulation hair growing device provided in the present disclosure is light and small, and is easy for users to carry and/or use; besides, the electrical stimulation hair growing device provided in Example 2 can convert the mechanical energy in external environment into electric energy by means of the electrical pulse module, so as to charge the power module, which can prolong the power supply time of the power module, as well as is energy-efficient and environmentally friendly; moreover, it is simple in structure and manufacturing process, and has low cost, so it is suitable for large-scale industrial production.

Both the electrical stimulation hair growing device in Example 1 and that in Example 2 of the present disclosure can be detachable and/or portable. In this way, it is convenient for the user to carry and/or use, thereby ensuring that the electrical stimulation hair growing device of the present disclosure can be used by the user in most occasions.

FIG. 9 shows a structure diagram of the electrical stimulation hair growing device according to one example of the present disclosure. As shown in FIG. 9, the electrical stimulation hair growing device in this example comprises: a power supply module 10b, a central control module 20b, an electrical pulse generating module 30b, and an electrical stimulation module 40b.

The power supply module 10b respectively supplies power to the central control module 20b and the electrical pulse generating module 30b by means of electrical connection, the electrical pulse generating module 30b is in electrical connection to the electrical stimulation module 40b, and the electrical stimulation module 40b is in contact with a skin surface of an area in need of hair growing. The electrical pulse generating module 30b is controlled by the central control module 20b to generate regular electrical pulse signals and transmit the regular electrical pulse signals to the electrical stimulation module 40b, so that the electrical stimulation module 40b applies the electrical pulse signals to the area in need of hair growing for electrical stimulation.

The central control module 20b and the electrical pulse generating module 30b are respectively in electrical connection to the power supply module 10b, so as to receive the power supplied by the power supply module 10b for work. The central control module 20b controls the electrical pulse generating module 30b to generate regular electrical pulse signals, which are transmitted to the electrical stimulation module 40b through the electrical connection between the electrical pulse generating module 30b and the electrical stimulation module 40b. The electrical stimulation module 40b applies electrical stimulation to the skin surface, with which the electrical stimulation module is in contact, of the area in need of hair growing, so as to activate epidermal hair follicles of a human head and promote hair growth.

The electrical stimulation hair growing device in this example can be directly placed at the skin surface of the area in need of hair growing for work, in which case the power supply module 10b may be a linear power supply, a switch power supply or other forms; the electrical stimulation hair growing device in this example can also be provided on an existing hat or cap, or headgear, or helmet, in which case the power supply module 10b may be a charging power supply or other forms. For example, the electrical stimulation module 40b can be provided inside a hat or cap close to the skin surface of the area in need of hair growing, and the central control module 20b and the power supply module 10b in the form of a charging power supply can be provided outside the hat or cap for decoration, thereby realizing portable operation of the electrical stimulation hair growing device.

As an optional embodiment, the central control module 20b comprises a parameter adjustment module 21b. The parameter adjustment module 21b is in electrical connection to the electrical pulse generating module 30b, and is used for adjusting at least one of voltage, current, waveform, amplitude, width and frequency of the electrical pulse signal generated by the electrical pulse generating module 30b.

FIG. 10 shows a structure diagram of the electrical stimulation hair growing device according to another example of the present disclosure. As shown in FIG. 10, based on the first example shown in FIG. 9, the electrical stimulation hair growing device in this example further comprises a remote control module 50b, and the central control module 20b of the electrical stimulation hair growing device in this example further comprises a parameter adjustment module 21b and a wireless transceiver module 22b.

The parameter adjustment module 21b is in electrical connection respectively to the electrical pulse generating module 30b and the wireless transceiver module 22b, and the wireless transceiver module 22b is in wireless connection to the remote control module 50b.

When the wireless connection between the wireless transceiver module 22b and the remote control module 50b doesn't work, the user directly controls the parameter adjustment module 21b to adjust the electrical pulse signals generated by the electrical pulse generating module 30b. Electrical pulse signal parameters adjusted by the parameter adjustment module 21b comprises at least one of voltage, current, waveform, amplitude, width and frequency. For example, the user can adjust the electrical pulse signals to be in a waveform of rectangular wave, trapezoidal wave, sharp pulse, sawtooth wave, step wave and so on, adjust maximum voltage or current intensity, adjust the high-level duration, and adjust the pulse frequency, so that the regular electrical pulse signals generated by the electrical pulse generating module 30b fit with activation parameters of epidermal hair follicles of a human head.

When the wireless connection between the wireless transceiver module 22b and the remote control module 50b works, the user controls the remote control module 50b to give an instruction to the wireless transceiver module 22b, so that the parameter adjustment module 21b makes adjustment according to the instruction, and controls the electrical pulse generating module 30b to generate corresponding regular electrical pulse signals. The instruction given by the remote control module 50b may be, but is not limited to, an instruction to directly control voltage, current, waveform, amplitude, width, frequency and other parameters of the electrical pulse signals, and may also be “High-intensity mode”, “Mild mode”, “Minor mode” and other illustrative scene instructions. The parameter adjustment module 21b adjusts parameters of the electrical pulse signals according to the scene instruction received by the wireless transceiver module 22b, so as to control the electrical pulse generating module 30b to generate corresponding regular electrical pulse signals. The remote control module 50b may be, but is not limited to, a mobile terminal, an IR remote control, a PC or a third-party software system and so on. One skilled in the art can flexibly select the form of the remote control module 50b according to actual necessities, for example, a remote control software of the electrical stimulation hair growing device can be installed in an intelligent watch, and, for another example, an APP of the electrical stimulation hair growing device can be installed in a mobile terminal.

Using the electrical stimulation hair growing device in this example, the user can directly control parameters of the electrical pulse signals, as well as give an instruction at a long distance through the remote control module 50b, as a result of which the electrical stimulation hair growing device can be used more flexibly.

As an optional embodiment, the power supply module 10b comprises a charging module 11b and an energy storage module 12b. The energy storage module 12b is in electrical connection respectively to the charging module 11b, the central control module 20b and the electrical pulse generating module 30b, receives electric energy through the charging module 11b, and supplies power to the central control module 20b and the electrical pulse generating module 30b.

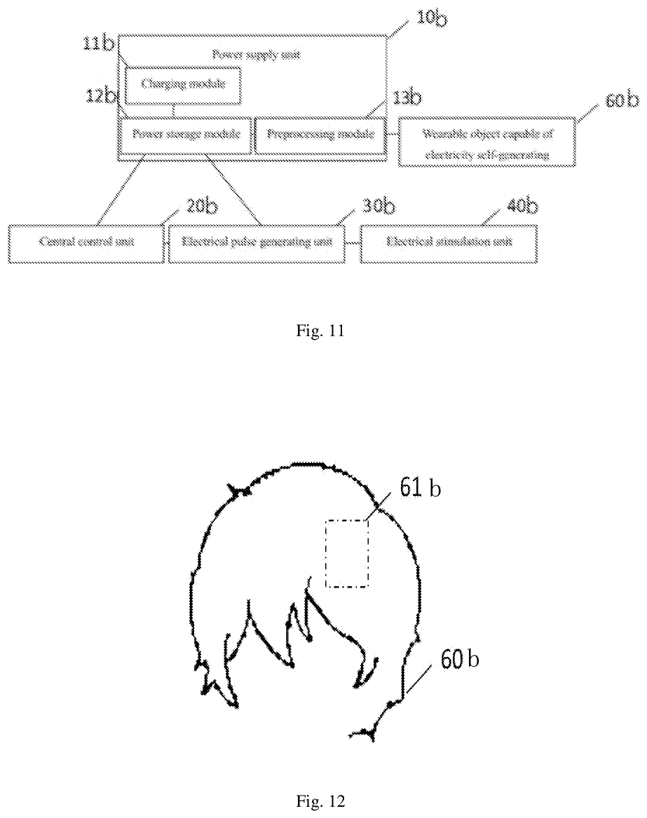

FIG. 11 shows a structure diagram of the electrical stimulation hair growing device according to another example of the present disclosure. As shown in FIG. 11, based on the first example shown in FIG. 9, the electrical stimulation hair growing device in this example further comprises a wearable object 60b capable of electricity self-generating, and the power supply module 10b of the electrical stimulation hair growing device in this example further comprises a charging module 11b, an energy storage module 12b and a preprocessing module 13b.

The energy storage module 12b is in electrical connection respectively to the charging module 11b, the central control module 20b and the electrical pulse generating module 30b, and is in electrical connection, through the preprocessing module 13b, to the wearable object 60b capable of electricity self-generating.

The charging module 11b may be, but is not limited to, a Micro USB interface, a USB Type C interface, a Lightning interface and other common charging forms in the prior art. Electric energy can be stored in the energy storage module 12b through connection of the charging module 11b to a mobile phone, a power bank, a DC power supply and so on, so that power can be supplied to the central control module 20b and the electrical pulse generating module 30b.

The wearable object 60b capable of electricity self-generating can convert force acted thereon into electrical signals transmitted to the preprocessing module 13b, and electrical signals are preprocessed by the preprocessing module 13b and are transmitted to the energy storage module 12b, so as to provide power to the central control module 20b and the electrical pulse generating module 30b. In other words, the electrical stimulation hair growing device in this example can charge the energy storage module 12b through the charging module 11b so as to provide electric energy, and the energy storage module 12b can also be charged at any time through the wearable object 60b capable of electricity self-generating when charging is not available, so as to provide electric energy.

The preprocessing module 13b may comprise, but is not limited to, a rectification part, an amplification part and a filtration part. The wearable object 60b capable of electricity self-generating, the rectification part, the amplification part, the filtration part and the energy storage module 12b are electrically in series and in sequence. The rectification part can rectify the electrical signals output by the wearable object 60b capable of electricity self-generating, the amplification part can amplify the electrical signals output by the rectification part, and the filtration part can filter out interference noise from the electrical signals output by the amplification part. Of course, the afore-mentioned parts comprised in the preprocessing module 13b are optional, and can be selected by one skilled in the art according to actual necessities; these parts are only required to enable the preprocessing module 13b to process the electrical signals output by the wearable object 60b capable of electricity self-generating and transmit the processed electrical signals to the energy storage module 12b, and will not be limited here.

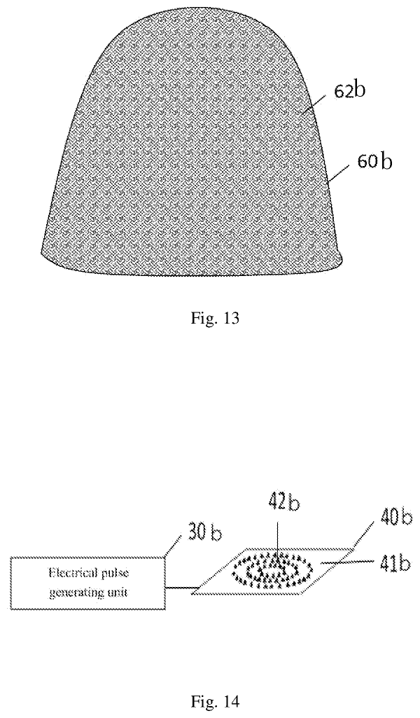

The wearable object 60b capable of electricity self-generating may be at least one of a hat or cap, a wig, a helmet, a turban, a collar, a sheet or other forms. The wearable object 60b capable of electricity self-generating may comprise a triboelectric generator, and/or a piezoelectric generator, and/or a pressure inductive cable based on a triboelectric generator and/or a piezoelectric generator, and/or a piezoelectric and triboelectric hybrid generator. For instance, the wearable object 60b capable of electricity self-generating may be a hat or cap, a wig, a helmet, a turban, a collar, a sheet and so on in the prior art that is additionally provided with a triboelectric generator, and/or a piezoelectric generator, and/or a pressure inductive cable based on a triboelectric generator and/or a piezoelectric generator, and/or a piezoelectric and triboelectric hybrid generator; the wearable object 60b capable of electricity self-generating may also be a hat or cap, a wig, a helmet, a turban, a collar, a sheet and so on that is made of a triboelectric generator, and/or a piezoelectric generator, and/or a pressure inductive cable based on a triboelectric generator and/or a piezoelectric generator, and/or a piezoelectric and triboelectric hybrid generator. As shown in FIG. 12, the wearable object 60b capable of electricity self-generating may be in form of a wig in the prior art that is additional provided with a layered film-type triboelectric generator 61b. The layered film-type triboelectric generator 61b is hidden inside the wig; as shown in FIG. 13, the wearable object 60b capable of electricity self-generating may be in form of a hat in the prior art that is made of a friction part-based knitted triboelectric generator 62b. When the wearable object 60b capable of electricity self-generating is worn by a user, even a casual turning or nodding by the user can convert the force acted on the wearable object 60b capable of electricity self-generating into electrical signals output to the preprocessing module 13b, and the preprocessing module 13b preprocesses received electrical signals and then transmits them to the energy storage module 12b.

When the wearable object 60b capable of electricity self-generating is in form of a hat or cap, a wig, a helmet, a turban and so on that can be worn on a head, since the electrical stimulation module 40b is also in contact with the skin surface of the area in need of hair growing, it is preferable that an insulating layer is provided between the electrical stimulation module 40b and the wearable object 60b capable of electricity self-generating, so as to avoid conductive interference that may happen between the wearable object 60b capable of electricity self-generating and the electrical stimulation module 40b.

For better understanding of the wearable object 60b capable of electricity self-generating by one skilled in the art, it will be introduced in detail below.

It is a case that the triboelectric generator may comprise at least one layered film-type triboelectric generator 61b. Each of the at least one layered film-type triboelectric generator 61b comprises at least two surfaces constituting friction interfaces, and at least one of the two surfaces constituting friction interfaces has an output end of electrical signals of the film-type triboelectric generator.

The triboelectric generator may be, but is not limited to, any film-type triboelectric generator having a three-layer structure, a four-layer structure, a five-layer intermediate film structure or a five-layer intermediate electrode structure. See the afore-mentioned description for details about the triboelectric generator.

It is a case that the piezoelectric generator can be any one of a zinc oxide piezoelectric generator, a PZT piezoelectric generator, or a PVDF piezoelectric generator.

A shielding layer can be provided outside the wearable object 60b capable of electricity self-generating, so as to avoid electromagnetic interference from external environment; and a protective layer can be provided outside the wearable object 60b capable of electricity self-generating, so as to avoid the influence of external environmental factors. For example, a shielding layer can be provided outside the layered film-type triboelectric generator 61b, and the layered film-type triboelectric generator 61b provided with a shielding layer is additionally provided and is hidden inside of the wig. For another example, a protective layer can be provided outside the friction part-based knitted triboelectric generator 62b, thereby forming a hat-shaped wearable object 60b capable of electricity self-generating. This can be flexibly provided by one skilled in the art according to actual situations.

FIG. 14 shows a structure diagram of the electrical stimulation hair growing device according to another example of the present disclosure. As shown in FIG. 14, the electrical stimulation module 40b of the electrical stimulation hair growing device in this example comprises at least one electrode 41b. The at least one electrode 41b is provided with at least one conductive bulge 42b that is provided on the surface at one side of the at least one electrode 41b close to the area in need of hair growing.

According to the position and/or size of the area in need of hair growing, the position and/or size of the at least one electrode 41b can be flexibly provided by one skilled in the art. The number, size, shape, providing and/or shape formed after providing of the at least one conductive bulge 42b on the at least one electrode 41b can also be selected by one skilled in the art according to actual necessities. As an example, a plurality of conductive bulges 42b is provided on the surface at one side of a plurality of electrodes 41b close to the area in need of hair growing, and the plurality of conductive bulges 42b is provided in a circular array. As an example, a plurality of conductive bulges 42b matching the size of hair follicles of the object in need of hair growing is provided on the surface at one side of one electrode 41b close to the area in need of hair growing. The electrode 41b and the conductive bulge 42b are flexibly provided so that the electrical stimulation module 40b is more suitable for the distribution and size of hair follicles of the object in need of hair growing, as a result, electrical pulses can be better applied to the area in need of hair growing for electrical stimulation.

The electrical stimulation hair growing device provided in the present disclosure accurately adjusts the electrical pulse generating module 30b through the central control module 20b so as to generate regular electrical pulse signals transmitted to the electrical stimulation module 40b, performs remote intelligent control through the remote control module 50b, performs energy storage and charging through the charging module 11b, and provides, through the wearable object 60b capable of electricity self-generating, electric energy support at any time when charging is not available. The electrical stimulation hair growing device provided in the present disclosure can be directly placed on the skin surface of the area in need of hair growing, or can be hidden inside an existing hat or cap, or wig, or can directly serve as a wearable device when it is comprised in the wearable object 60b capable of electricity self-generating. The electrical stimulation hair growing device provided in the present disclosure makes electrical pulse signal adjustment more accurate, and also enables the wearing and using more flexible, as a result of which the electrical stimulation hair growing device can be used in much more scenes.

Afore-mentioned are merely embodiments of the present disclosure. Taught by the present disclosure, one skilled in the art can make other improvements or modifications on the basis of the above-mentioned examples. It should be understood by one skilled in the art that the afore-mentioned illustration is merely for better explanation of the present disclosure, and the scope of protection of the present disclosure should follow the scope of protection of claims.

What is claimed is:

1. An electrical stimulation hair growing device, comprising: an energy providing module, an electrical pulse generating module and an electrical stimulation module, wherein:
the energy providing module provides electric or mechanical energy to the electrical pulse generating module respectively through electrical or mechanical connection, the electrical pulse generating module is in electrical connection to the electrical stimulation module, and the electrical stimulation module is in contact with a skin surface of an area in need of hair growing; and
the electrical pulse generating module generates regular electrical pulse signals by means of the electric or mechanical energy and transmits the regular electrical pulse signals to the electrical stimulation module, so that the electrical stimulation module applies the electrical pulse signals to the area in need of hair growing for electrical stimulation.

2. The electrical stimulation hair growing device according to claim 1, wherein the energy providing module comprises a vibration module,
the vibration module is used for generating vibration with a predetermined frequency and a predetermined amplitude; and
the electrical pulse module is provided in contact with the vibration module, and is used for converting vibration acted on the electrical pulse module by the vibration module into electrical pulses and outputting the electrical pulses, and/or for converting mechanical energy acted on the electrical pulse module by external environment into electrical pulses and outputting the electrical pulses.

3. The electrical stimulation hair growing device according to claim 2, wherein the vibration module further comprises: a power module, a driver module, and at least one vibration part;
the power module is connected to the driver module, and is used for providing electric energy to the driver module;
the driver module is connected to the at least one vibration part, and is used for controlling the at least one vibration part to vibrate at the predetermined frequency and the predetermined amplitude; and
the at least one vibration part is provided in contact with the electrical pulse module, and is used for generating vibration having the predetermined frequency and the predetermined amplitude so as to act the vibration on the electrical pulse module.

4. The electrical stimulation hair growing device according to claim 3, wherein a transmission insulating part for transmitting vibration and for avoiding conduction of the at least one vibration part and the electrical pulse module is further provided between the at least one vibration part and the electrical pulse module.

5. The electrical stimulation hair growing device according to claim 3, wherein the vibration part is a rotor motor, a longitudinal linear motor or a transverse linear motor.

6. The electrical stimulation hair growing device according to claim 5, wherein the number of vibration parts is multiple, the multiple vibration parts comprise the same number of the longitudinal linear motors and the transverse linear motors, and the longitudinal linear motors and the transverse linear motors are uniformly distributed in arrays on the electrical pulse module.

7. The electrical stimulation hair growing device according to claim 1, wherein the energy providing module comprises a power supply module and a central control module,
the power supply module provides power to the central control module and the electrical pulse generating module respectively by means of electrical connection; and
the electrical pulse generating module generates regular electrical pulse signals by means of control of the central control module and transmits the regular electrical pulse signals to the electrical stimulation module.

8. The electrical stimulation hair growing device according to claim 7, wherein the central control module comprises a parameter adjustment module;
the parameter adjustment module is in electrical connection to the electrical pulse generating module; and
the parameter adjustment module is used for adjusting at least one of voltage, current, waveform, amplitude, width and frequency of the electrical pulse signal generated by the electrical pulse generating module.

9. The electrical stimulation hair growing device according to claim 7, wherein the power supply module comprises a charging module and an energy storage module;
the energy storage module is in electrical connection respectively to the charging module, the central control module and the electrical pulse generates module; and
the energy storage module receives electric energy through the charging module, and provides power to the central control module and the electrical pulse generating module.

10. The electrical stimulation hair growing device according to claim 9, wherein the device comprises a wearable object capable of electricity self-generating, and the power supply module comprises a preprocessing module;
the preprocessing module is in electrical connection respectively to the wearable object capable of electricity self-generating and the energy storage module; and
the wearable object capable of electricity self-generating is used for converting force acted on the wearable object into electrical signals and transmitting the electrical signals to the preprocessing module, and the preprocessing module preprocesses received electrical signals and transmits preprocessed electrical signals to the energy storage module.

11. The electrical stimulation hair growing device according to claim 1, wherein the electrical stimulation module further comprises a plurality of electrodes; and at least one electrical stimulation bulge is provided on a side surface where the plurality of electrodes is in contact with the area in need of hair growing.

12. The electrical stimulation hair growing device according to claim 1, wherein the electrical pulse module comprises: a triboelectric generator, and/or a piezoelectric generator, and/or a pressure inductive cable based on a triboelectric generator and/or a piezoelectric generator, and/or a piezoelectric and triboelectric hybrid generator.

13. The electrical stimulation hair growing device according to claim 12, wherein the triboelectric generator is a triboelectricity generating film; and/or the piezoelectric generator is a piezoelectricity generating film.

14. The electrical stimulation hair growing device according to claim 12, wherein the triboelectric generator comprises a friction part-based knitted triboelectric generator;
the friction part-based knitted triboelectric generator comprising: at least one first friction part and at least one second friction part; wherein:
the at least one first friction part and the at least one second friction part are knitted with each other to form the friction part-based knitted triboelectric generator, and at least one of the at least one first friction part and the at least one second friction part comprises a friction interface capable of contacting friction; and
the at least one first friction part and/or the at least one second friction part have an electrical signal output end of the friction part-based knitted triboelectric generator.

15. The electrical stimulation hair growing device according to claim 12, wherein the pressure inductive cable based on a triboelectric generator and/or a piezoelectric generator is knitted to form the electrical pulse module having a predetermined shape.

16. The electrical stimulation hair growing device according to claim 14, wherein the at least one first friction part and the at least one second friction part are knitted to form the electrical pulse module having a predetermined shape.

17. The electrical stimulation hair growing device according to claim 15, wherein the predetermined shape is a hat or cap shape, a headgear shape, a turban shape, a collar shape or a sheet shape.

18. The electrical stimulation hair growing device according to claim 1, wherein the electrical stimulation hair growing device further comprises a wearable body; the wearable body is worn on an object in needed of hair growing, and is provided with the at least one vibration module, the electrical pulse module and/or the electrical stimulation module.

19. The electrical stimulation hair growing device according to claim 18, wherein the wearable body is a hat or cap, a headgear, a turban, a collar or a sheet.

20. The electrical stimulation hair growing device according to claim 1, wherein the electrical stimulation hair growing device is a detachable and/or portable electrical stimulation hair growing device.
